(19) United States
(12) Patent Application Publication
(10) Pub. No.: US 2021/0054339 A1
(43) Pub. Date: Feb. 25, 2021

(54) METHOD FOR THE PRODUCTION OF CELLULAR PARTICULATE WITH ANTITUMOR ACTIVITY

(71) Applicant: Rigenerand S.R.L., Medolla (IT)

(72) Inventors: Filippo Rossignoli, Verona (IT); Carlotta Spano, Modena (IT); Giulia Grisendi, Reggio Emilia (IT); Olivia Candini, Crevalcore (IT); Massimo Dominici, Ferrara (IT)

(21) Appl. No.: 17/050,397

(22) PCT Filed: Apr. 24, 2019
(86) PCT No.: PCT/IT2019/050081

§ 371 Date: Oct. 23, 2020

(30) Foreign Application Priority Data
Apr. 26, 2018 (IT) 102018000004901

(51) Int. Cl. C12N 5/077; A61K 35/35

(52) U.S. Cl. CPC C12N 5/0653; C12N 2502/99; A61K 35/35

(57) ABSTRACT

Method for the production of cellular particulate with antitumor activity, which comprises isolating the cellular particulate originating from a cell population with a phenotype that can be attributed to human adipose tissue derived pericytes (AD-PC) that express antitumor TRAIL.

### FIELD OF THE INVENTION

The present invention concerns a method for the production of cellular particulate with antitumor activity within a cell therapy approach, which can be implemented ex vivo.

### BACKGROUND OF THE INVENTION

It is known that adult progenitor cells, including a cell population with a phenotype attributable to human adipose tissue-derived pericytes (hereafter AD-PC for short), can be used as a vehicle to carry bio-active molecules.

In particular, AD-PCs can carry so-called “death ligands”, that is, a family of molecules belonging to the Tumor Necrosis Factor superfamily.

Among these, the Tumor necrosis factor-Related Apoptosis-Inducing Ligand (hereafter TRAIL for short) molecule can induce cell death in diseased tissues, and therefore is particularly interesting for its possible use in certain biomedical fields.

From European patent EP2424979 a method to produce medications for the treatment of tumors is known, which comprises preparing a retroviral vector which encodes a soluble variant of the TRAIL molecule, and which stably transfects adipose pericytes (AD-PC).

AD-PCs that carry TRAIL have shown cytotoxic activity in vitro and in vivo against some tumors and are therefore particularly interesting in the context of cancer treatments.

Furthermore, the term “cellular particulate” indicates a product of cellular activity consisting of particles that are released spontaneously outside the cell. These particles are delimited by a closed circular membrane consisting entirely or partially of phospholipids. Furthermore, they are characterized by a diameter size comprised between 40 nm and 5000 nm.

The cellular particulate can originate from protuberances of the plasma membrane which subsequently detach from it.

Alternatively, the cellular particulate can originate within intracellular organelles known as endosomes, and subsequently be released outside the cell by fusion of the endosomal membrane with the plasma membrane.

Alternatively, the cellular particulate can originate from protrusions of the plasma membrane during the process of cellular apoptosis.

It is known that the cellular particulate can include, in the lumen or in the membrane of the particles of which it consists, molecules deriving from the cell that produced it.

These molecules are then conveyed outside the cell together with the cellular particulate.

However, this state of the art is affected by some problems.

A first problem is that the safety of treatments that provide to inoculate living cells carrying bioactive molecules in an organism for therapeutic purposes is still being debated, for the reason that the fate of the inoculated cells cannot be controlled in advance, that is, it is not known how cells behave after inoculation.

The main uncertainties concern their potential for uncontrolled expansion, which can cause damage to healthy organs and alterations of physiological functions.

A second problem is that inoculating living cells carrying bioactive molecules in an organism for therapeutic purposes can trigger rejection reactions by the receiving organism that can nullify the therapy itself.

A third problem is that the use of living cells carrying bioactive molecules that have to be infused requires the production of a large number of cells with consequently high costs.

One purpose of the invention is to improve the state of the art.

Another purpose of the invention is to provide a method for the production of cellular particulate derived from TRAIL-producing AD-PCs (AD-PC-TRAIL) for antitumor purposes.

Another purpose of the invention is to provide a method for the production of cellular particulate derived from AD-PC-TRAIL without compromising the antitumor action of TRAIL.

Another purpose of the invention is to provide a method for the production of cellular particulate derived from AD-PC-TRAIL that reduces the simultaneous administration of AD-PC-TRAIL limiting every undesired side effect for the organism attributable to the AD-PCs themselves.

Another purpose of the invention is to provide a method for the production of cellular particulate derived from AD-PC-TRAIL which reduces the simultaneous administration of AD-PC-TRAIL, making the administration of TRAIL substantially economical.

According to one aspect, the invention provides a method for the production of cellular particulate with antitumor activity, according to the characteristics of claim 1.

According to another aspect of the invention, a cellular particulate is provided according to the characteristics of claim 5.

### BRIEF DESCRIPTION OF THE DRAWINGS

Other characteristics and advantages of the invention will become more apparent from the following description of an embodiment of a method for the production of cellular particulate with antitumor activity, given as a non-restrictive example with reference to the attached drawings wherein:

FIG. 1 is a dot plot showing a population of calibrated beads of a known diameter and comprised between 500 nm and 3000 nm analyzed with flow cytometry. The horizontal line “A” separates the background noise of the instrument (below the line) from the points representing the data obtained (above the line);

FIG. 2 is a dot plot showing the isolated cellular particulate analyzed with flow cytometry. The horizontal line “A” separates the background noise of the instrument (below the line) from the points representing the data obtained (above the line);

FIG. 3 is a histogram showing the fluorescence of the cellular particulate isolated from AD-PC and stained with the carboxyfluorescein diacetate succinimidyl ester (CFDA-SE) molecule.

In detail, line “B” divides a left field in which a lower fluorescence intensity was found, comparable to that of cellular particulate not stained with CFDA-SE, from a right field in which a greater fluorescence intensity was found.

FIG. 4 is a table showing the TRAIL concentrations detected in the AD-PC derived cellular particulate modified with an empty vector and therefore not expressing TRAIL (AD-PC-EMPTY) or AD-PC-TRAIL expressed in pg/million cells.

FIG. 5 is a diagram of a cytotoxicity assay among BXPC3 tumor cells incubated with cellular particulate derived from AD-PC-EMPTY or AD-PC-TRAIL;

FIG. 6 is a diagram of a cytotoxicity assay among A673 tumor cells incubated with cellular particulate derived from AD-PC-EMPTY or AD-PC-TRAIL;

FIG. 7 is a diagram of a Caspase 8 activation assay on A673 cells incubated with AD-PC-TRAIL derived cellular particulate, compared with control A673 cells.

### DETAILED DESCRIPTION OF SOME EMBODIMENTS

The invention concerns a method for the production of cellular particulate, in particular derived from AD-PC-TRAIL, which has been found to have antitumor activity.

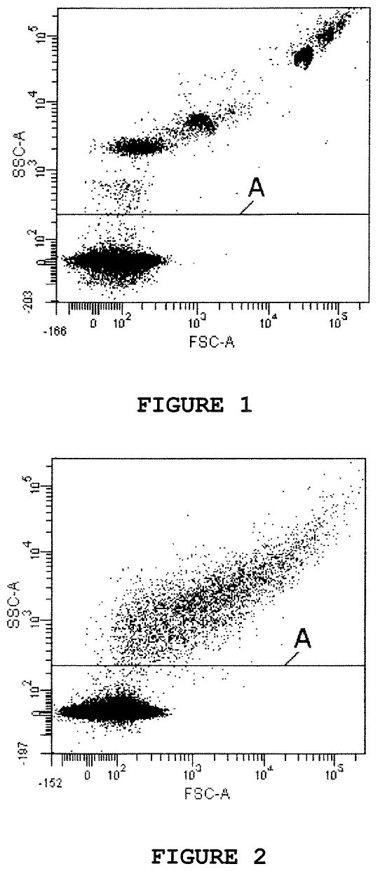

With reference to FIG. 1, this shows the flow cytometry analysis based on the Forward Scatter (FSC) and Side Scatter (SSC) morphological parameters of a sample of calibrated beads of known size comprised between 500 nm and 3000 nm.

With reference to FIG. 2, this shows the flow cytometry analysis of the cellular particulate isolated from AD-PC with reference to the FSC and SSC morphological parameters.

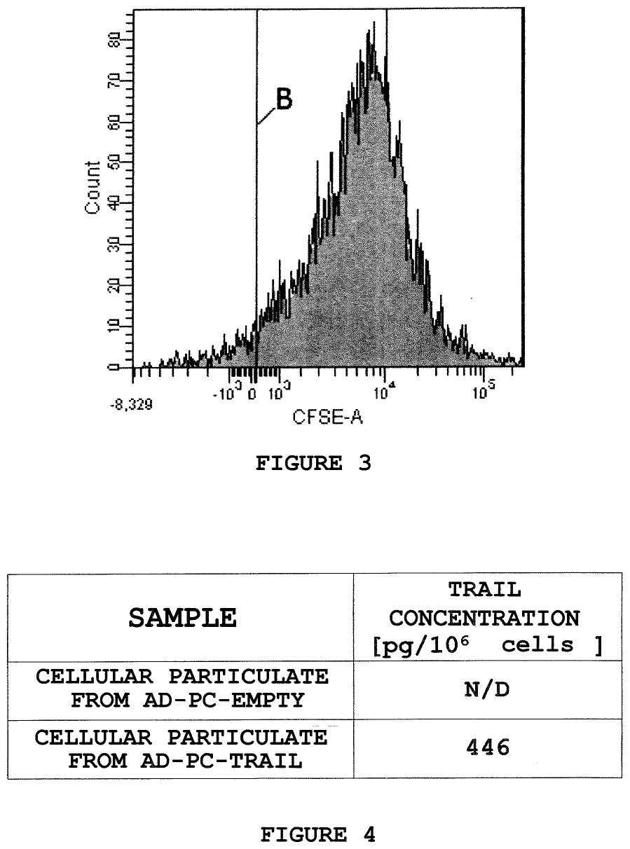

With reference to FIG. 3, this shows the emission of fluorescent light of the cellular particulate isolated from AD-PC after staining with CFDA-SE.

In particular, 95.9% of cellular particulate tested positive for fluorescence.

With reference to the table in FIG. 4, this shows the TRAIL concentrations detected in the cellular particulate derived from AD-PC-EMPTY or AD-PC-TRAIL, by means of an enzyme-linked immunoabsorbent assay (ELISA).

In particular, it is noted that no presence of TRAIL released from the AD-PC-EMPTY derived cellular particulate was detected, while 446 pg of TRAIL obtained from the cellular particulate derived from 1 million AD-PC-TRAIL were measured.

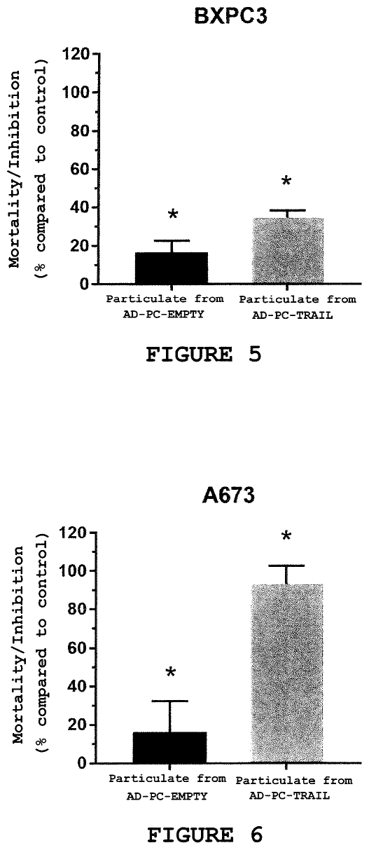

With reference to FIG. 5, the cytotoxicity assay shows that the AD-PC-TRAIL derived cellular particulate is able to cause a cytotoxic effect on the BXPC3 tumor cell line.

In particular, the mortality of BXPC3 cells measured is 34.6±3.6% when the cells are incubated with AD-PC-TRAIL derived cellular particulate, against a mortality of 16.5±6.2% when the cells are incubated with AD-PC-EMPTY derived cellular particulate. Using a T-test statistical test, a statistically significant difference was found between the groups characterized by a value of p<0.05 (symbolized with * in the drawing).

The data are represented as mean±SEM (Standard Error of the Mean) and the same representation is used below in the other drawings.

With reference to FIG. 6, the cytotoxicity assay shows that the AD-PC-TRAIL derived cellular particulate is able to cause a cytotoxic effect on the tumor cell line A673.

In particular, the mortality detected is 93.1% when the cells are incubated with AD-PC-TRAIL derived cellular particulate, against a mortality of 16.1±16.2% when the cells are incubated with AD-PC-EMPTY derived cellular particulate.

Using a T-test statistical test, a statistically significant difference was found between the groups characterized by a value of p<0.01 (symbolized with * in the drawing).

The data are represented as mean±SEM.

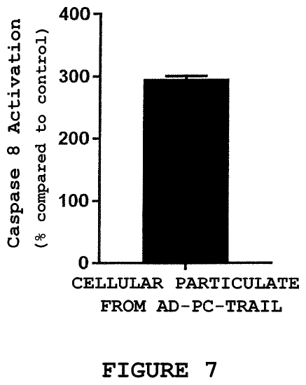

With reference to FIG. 7, this shows the percentage of activation of caspase 8 on A673 cells after incubation with AD-PC-TRAIL derived cellular particulate with respect to the reference control. The treatment has determined a caspase activation of 296±5% higher than in the control.

Using a T-test statistical test, a statistically significant difference was found with respect to the control, characterized by a value of p<0.001. The data are represented as mean±SEM.

### EXAMPLE 1

### Isolation of the Cellular Particulate Produced by AD-PC

The method for isolating cellular particulate produced by AD-PC is as follows.

The AD-PCs kept in culture, having reached a confluence of about 80%, were washed with PBS (produced by Biochrom GmbH) and the culture medium was replaced with αMEM medium (produced by Thermo Fisher Scientific Inc.) containing 1% of L-glutamine (produced by Lonza) and 1% penicillin/streptomycin (10000 U/mL penicillin, 10 mg/mL streptomycin in 0.9% NaCl, produced by PAA Laboratories).

The AD-PCs were then kept in culture in an incubator for 48 hours before proceeding with the collection of the supernatant.

The supernatant collected from the cell cultures of AD-PC-TRAIL and AD-PC-EMPTY was subjected to a differential centrifugation treatment, which provides an initial centrifugation at 2,000 g for 20 minutes at +4° C., a subsequent filtration by gravity of the supernatant with a 0.8 μm filter, and a further centrifugation at 20,000 g for 40 minutes at +4° C.

At the end of the phases the pellet containing the cellular particulate was resuspended in the most appropriate medium for subsequent applications.

### Display of Cellular Particulate in Flow Cytometry

The cellular particulate obtained with the previously described isolation protocol was resuspended in PBS containing 0.1% of BSA (Bovine Serum Albumin, produced by Sigma) and stained with 1 μL of CFDA-SE (CellTrace CFSE Cell Proliferation Kit, produced by Invitrogen) before being displayed on the flow cytometer (BD FACS ARIA III, produced by Becton Dickinson).

The optimal parameters of FSC and SSC to display the MVs were set using beads of known sizes (Megamix, produced by Biocytex).

### Results of Example 1

The cellular particulate matter was isolated from the culture medium conditioned by AD-PC-EMPTY and AD-PC-TRAIL through two centrifugation passes separated by a filtration with a 0.8 μm filter and displayed on the flow cytometer.

This protocol has led to the isolation of a population of particles with sizes comprised between 40 nm and 5000 nm, and in particular approximately comprised between 500 nm and 3000 nm, as shown by the comparison with the sizes of Megamix beads of known size (see FIG. 1 and FIG. 2).

The staining with CFDA-SE allowed a better display of the MVs in flow cytometry (see FIG. 3).

This is a dye able to penetrate into the cell cytoplasm where it is converted, by intracellular esterase, into the succinimidyl ester compound of carboxyfluorescein (CFSE), which is retained inside the cells bound to intracellular proteins and emits a luminous fluorescent signal that can be detected.

Due to this characteristic, CFDA-SE allows to distinguish closed structures, coated by membrane, in this case the cellular particulate, from cellular debris.

### EXAMPLE 2

Quantifying the amount of TRAIL present in the cellular particulate. The cellular particulate derived from AD-PC-EMPTY and AD-PC-TRAIL was subjected to the ELISA test (Quantikine ELISA, produced by R&D Systems) to determine TRAIL concentration.

In particular, the cellular particulate isolated with the isolation protocol previously described in example 1 was resuspended in PBS containing 0.1% BSA and lysed with a suitable lysing solution (Lysis Buffer, produced by R&D Systems) before proceeding with the test, according to the manufacturer's protocol.

### Results of Example 2

The TRAIL content in the cellular particulate derived from AD-PC-EMPTY and AD-PC-TRAIL was quantified using the ELISA test after inducing the lysis of the particulate itself. The results did not show the presence of TRAIL in AD-PC-EMPTY derived samples, while a concentration of 446 pg was detected in the cellular particulate derived from 1 million AD-PC-TRAIL (see FIG. 4).

As is known, AD-PC-TRAIL produce a soluble TRAIL molecule lacking the structural elements necessary to insert the particles that form the cellular particulate into the membrane. Therefore, the TRAIL molecules detected are originally localized in the lumen of the particles that form the particulate.

### EXAMPLE 3

### Cytotoxicity Assays with Cellular Particulate on Tumor Cell Lines

The antitumor capacity of the cellular particulate from AD-PC-EMPTY and AD-PC-TRAIL was tested using the MTS assay (CellTiter96® AQueous One Solution Cell Proliferation Assay, produced by Promega) on cells of the tumor lines A673 and BXPC3.

For each sample analyzed, 5,000 tumor cells were seeded in a 96 well plate and kept in culture in a controlled atmosphere incubator (37° C., 5% CO₂).

The following day, the tumor cells culture medium was replaced with 100 μL of DMEM medium supplemented with 10% FBS and 1% penicillin/streptomycin, in which the intact cellular particulate obtained following the isolation protocol previously described in example 1 was resuspended.

After 24 hours, 20 μL of solution per MTS were added to each well and the plate was kept in incubation at 37° C. for 1 hour, before proceeding to measure the absorbance at 495 nm, proportional to the vitality and the metabolic activity, using the GloMax Discover instrument (produced by Promega).

All the experiments included a control of the basal mortality consisting of tumor cells in contact with the same medium but without cellular particulate.

To confirm the role of TRAIL in inducing cytotoxicity on A673 cells, the activity of caspase 8 was studied using the Caspase-Glo 8 Assay kit (produced by Promega).

In particular, tumor cells were seeded in a white multi-well plate at a concentration of 5,000 cells per well.

The following day, the culture medium was replaced with a fresh medium containing the intact AD-PC-TRAIL derived cellular particulate, isolated as previously described.

After 18 hours, caspase 8 activity was evaluated using the GloMax Discover instrument, according to the protocol provided by the manufacturer.

### Results of Example 3

The mortality induced on the BXPC3 and A673 tumor lines, after 24-hour incubation with cellular particulate derived from AD-PC-EMPTY and AD-PC-TRAIL cells, was detected by means of cytotoxicity assay with MTS.

Treatment of BXPC3 cells with AD-PC-TRAIL derived cellular particulate gave a mortality of 34.6±3.6%, significantly higher than that obtained with AD-PC-EMPTY derived cellular particulate, which instead did not show relevant cytotoxic effects (see FIG. 5).

On A673 cells, the mortality induced by the AD-PC-TRAIL derived cellular particulate was 93.1±9.6%, significantly higher than that obtained with AD-PC-EMPTY derived cellular particulate, which instead did not show significant cytotoxicity effects (see FIG. 6).

The role of TRAIL in the induction of tumor cell mortality was verified by quantifying the activation percentage of caspase 8, compared to an untreated control, after 18-hour incubation of the A673 tumor line with AD-PC-TRAIL derived cellular particulate.

The treatment determined a caspase activation of 296±5% higher than the control, supporting a cytotoxic effect mediated by TRAIL (see FIG. 7).

In practice it has been found that the invention achieves the intended aims.

The invention as conceived is susceptible to modifications and variations, all of which come within the scope of the inventive concept.

Furthermore, all the details can be replaced with other technically equivalent ones.

In practice, any materials, equipment and quantities can be used, according to requirements, without departing from the field of protection of the following claims.

1. Method for the production of cellular particulate with antitumor activity, wherein it comprises isolating the cellular particulate from a supernatant collected from a cell population culture expressing an antitumor soluble TRAIL transfected with a retroviral vector which has a human adipose tissue derived pericytes (AD-PC) phenotype.

2. Method as in claim 1, wherein said cell population with a human adipose tissue derived pericytes (AD-PC) phenotype is transfected in a stable manner.

3. Method as in claim 1, wherein said cells with a pericytes (PC) phenotype are generated from tissues chosen from: adipose tissue, osteo-medullary tissue, placenta, amniotic fluid, dental pulp, muscle tissue, cardiac tissue, umbilical cord, cutaneous tissue, pancreatic tissue, intestinal tissue, decidual endometrial tissue.

4. Method as in claim 1, wherein said population expressing TRAIL is selected from: autologous cells, allogenic cells, human cells, animal cells.

5. Cellular particulate, wherein it comprises particles that have a closed circular membrane formed by a double layer consisting partially or entirely of phospholipids.

6. Cellular particulate as in claim 5, wherein said particles have a maximum size comprised between 40 nm and 5000 nm.

7. Cellular particulate as in claim 6, wherein said particles have a maximum size comprised between 500 nm and 3000 nm.

8. Cellular particulate as in claim 5, wherein said particles have TRAIL in their lumen with anti-cancer activity.
